(19) United States
(12) Patent Application Publication
(10) Pub. No.: US 2026/0272789 A1
(43) Pub. Date: Sep. 17, 2026

(54) A DENTAL COMPOSITE AND METHOD OF PHOTO-INDUCED POLYMERISATION USING NOVEL CO-INITIATOR

(71) Applicant: SHIV NADAR (INSTITUTION OF EMINENCE DEEMED TO BE UNIVERSITY), Nagar (IN)

(72) Inventors: Ajoy KAPAT, Nagar (IN); Asrar AHMAD, Nagar (IN); Garvisha MITTAL, Nagar (IN)

(73) Assignee: SHIV NADAR (INSTITUTION OF EMINENCE DEEMED TO BE UNIVERSITY), Nagar (IN)

(21) Appl. No.: 19/474,317

(22) PCT Filed: Jun. 26, 2023
(86) PCT No.: PCT/IN2023/050616

§ 371 Date: Oct. 10, 2025

(30) Foreign Application Priority Data
Apr. 12, 2023 (IN) 202311027002

(51) Int. Cl. A61K 6/62; A61K 6/889

(52) U.S. Cl. CPC A61K 6/62; A61K 6/889

(57) ABSTRACT

The present invention discloses a novel dental composite comprising photo induced polymerization reaction product of a plurality of monomers, a photo initiator, and a co-initiator. The co-initiator of the dental composite is selected from the compounds of Formula 1, Formula 2, Formula 3, Formula 4, and Formula 5. The method of preparation of the dental composite involves a first monomer which is reacted with a second monomer along with a photo initiator and a co-initiator in the presence of a Blue LED light at room temperature to yield a dental composite mixture, which when subjected to light source results in polymerization of the dental composite for dental restoration. The dental composite of the present invention is utilized as filling material in dental restoration procedures.

### FIELD OF THE INVENTION

The present invention generally relates to the field of dentistry. Specifically, the present invention relates to dental composites. More specifically, the present invention relates to dental composite as filling material in dental restoration purposes and process thereof. More particularly, the present invention relates to the method of photo-induced radical polymerization of the dental composite using novel co-initiators.

### BACKGROUND OF THE INVENTION

In dentistry, a multitude of alternative methods for addressing dental abnormalities are currently on the market. In general, dental problems can be corrected using either restorative or prosthetic procedures. The development of viable solutions in the field of dental restoration has mainly been guided by the ease of implementation, availability and physico-chemical properties of the materials studied. Silver-mercury amalgams, still used today, have significant drawbacks for patients, including toxicity, poor aesthetics and lack of adherence to dental tissues. Direct repair composites, for example, highly filled materials with good mechanical qualities are little wear. Unfortunately, because of the large filler loading, these materials are fragile.

At past, materials utilized for dental restorations have primarily been acrylate or methacrylate polymers. In specific, a dental composite is a resin mixture or a combination of glass and polymers, it is extensively used in dental treatments such as cavity filling, repair, and aesthetic reshaping due to its durability and safety. Unlike metal fillings in the past, composite also has a significant aesthetic advantage. In particular, composites are well prepared utilizing the photo induced polymerization reaction, and they also comprise an initiator or co-initiator that plays a significant role in the photo induced polymerization reaction. More efficient initiators for type I are either prohibitively expensive or need a multistep chemical reaction for manufacture. Another disadvantage of these initiators is oxidative quenching, which is responsible for the low polymerization rate. Most typically utilized co-initiators in type II PRP reactions are either mutagenic or cause yellowing in nature. In most circumstances, a longer curing period and higher loading are required. There are few reports available in the literature about the existence of composite material and utility aspects of it.

U.S. Pat. No. 6,417,246B1 discloses a dental composite material, the invention claims a polymerizable dental composition, comprising a polymerizable resin composition, and a filler composition comprising nano structured colloidal silica particles, wherein the silica particles are bound to each other to form chains.

WO2006057908A1 discloses a dental composite restorative material, the invention claims a dental restorative material for use in for example, class 1 & 2 posterior cavity restorations. The inventive material includes a modified BisGMA resin. The invention also includes a method of restoring a damaged tooth that includes the application of bonding agent, applying energy such as by warming the material so that it can be extruded, and placing the material into the tooth cavity.

EP1050291A2A discloses a dental restorative composite, this invention claims a dental restorative composition comprising between about 10% by volume and about 70% by volume of a ground structural filler having a mean particle size between about 0.05 μm and about 0.50 μm, wherein the ground structural filler contains less than 50% by volume of particles above 0.5 μm in diameter, mean particle size, a polymerizable acrylic monomer, and a phosphoric acid ester dispersant.

WO2019132849 (A2) relates to restorative purpose acrylic dental composite filling material which are curable by light and are polymerizable, and which contains only β-tricalcium phosphate (β-TCP), nanocrystalline cellulose (NCC), hydroxy apatite particles/fibers/whiskers, Al—Sr—OF and Al—Sr—Si—OF and/or mixtures thereof as supportive phase system for conferring regenerative and antibacterial properties to composite filling materials, and relates to production method of said dental composite filling material. Acrylic dental composite filling material according to the disclosure comprises organic compound, photoinitiator and supportive phase system which are curable by light and are polymerizable, characterized by comprising SiO₂, SiC Silane, Si/Zr nanocluster, Si/Zr/Silane nanocluster, TiO₂, TiO₂/Silane, ZrCO₂, 3YSZ, b-tricalcium phosphate (b-TCP), nanocrystalline cellulose (NCC), hydroxyapatite particles/fibers/whiskers, Al—Sr—OF and Al—Sr—Si—OF and/or mixtures thereof as supportive phase system for conferring regenerative and antibacterial properties to composite filling materials.

Now-a-days, 3D printing technology, coating and dental restoration process are booming techniques and these three processes are extremely important in our daily life and PRP reaction is becoming popular day by day due to its operational simplicity, economically sustainable and enviro-friendly in nature. Hence, this process has very high economic potential or commercial application for the aforementioned technology.

Hence the present invention aims to include newly synthesized co-initiator having 6, 5, 6, 6-bicyclic, and 6,7,6 tricyclic scaffolds and to develop a novel dental composite as filling material for dental restoration purposes.

However, the composites mentioned in the state of art result in yellowing coloration and inability to avoid the yellow coloration with time, lack of convenience for subject, the initiator utilized in the existing composites can initiate oxidative quenching process and hence become inefficient. Hence, there is an urgent need in the state of art to develop a novel dental composite wherein the composite is formed from monomers, photo initiator and novel co-initiators via photo-induced radical polymerization and subsequently the polymerized product is employed as a filling material in dental restoration procedures. Moreover, the major aspect of the present invention is to develop an efficient co-initiator which avoids oxidative quenching and avoids yellow coloration thereby overcoming the above said drawbacks.

### Objectives of the Invention

The main objective of the present invention is to develop a novel dental composite.

Another objective of the present invention is to develop a novel dental composite as filling material in dental restoration purposes.

Another objective of the present invention is to develop a novel dental composite which is solvent free, stabilizes in high glass transition temperature.

Another objective of the present invention is to develop a dental composite comprising plurality of monomers, photo-initiator, and co-initiator.

Another objective of the present invention is to develop a dental composite having lower initiator and co-initiator loading, curing time and polymeric material to provide a dental composite that is hydrophilic, colorless.

Another objective of the present invention is to develop a novel co-initiator which can be synthesized via highly efficient conjugate addition reaction with few merits such as ease of purification, cheap commercially available starting point.

Yet another objective of the present invention is to develop a novel dental composite which can be polymerized up to 3 cm (depth of the polymerization), thereby employed for bulk filling.

Yet another objective of the present invention is to develop a novel dental composite which employed with generated polymeric material turned out to be colorless and yellowing was not observed over 10-months.

Yet another objective of the present invention is to develop a novel dental composite which has operational simplicity.

Yet another objective of the present invention is to develop a dental composite that results from a photo induced radical polymerization reaction technique.

Yet another objective of the present invention is to develop a chemical reaction room temperature to form dental composite.

Further objective of the present invention is to utilize the developed dental composite as filling material in dental restoration purposes.

### SUMMARY OF THE INVENTION

Accordingly, the present invention discloses a novel dental composite, comprising photo induced polymerization reaction product of
i. a plurality of monomers,
ii. a photo initiator, and
iii. a co-initiator,
wherein the co-initiator is selected from the compounds of Formula 1, Formula 2, Formula 3, Formula 4 and Formula 5,

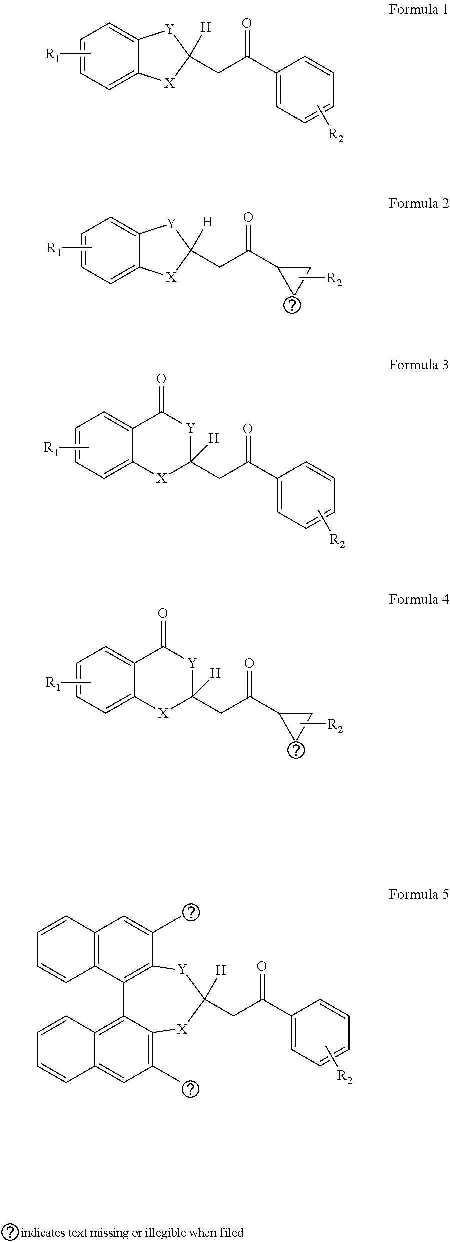

In Formula 1 and Formula 3,
X is selected from O, S, N, Se and Te;
Y is selected from O, S, N, Se and Te;
R₁ is selected from the group comprising of Hydro, alkyl, branched chain alkyl, aryl, tert-butyl, methyl, cyano, o-methyl, nitro and also comprises tert-Butyloxycarbonyl protected amine (BocHN);
R₂ is selected from group comprising of OMe, Me, CN, Methyl, Cl, F, Br, NO₂, OPh, OTs, allylic and the like;

In Formula 2 and Formula 4,
X is selected from O, S, N, Se and Te;
Y is selected from O, S, N, Se and Te;
wherein n=0,1,2,3,4,5,6
R₁ is selected from the group comprising of Hydro, alkyl, branched chain alkyl, aryl, tert-butyl, methyl, cyano, o-methyl, nitro and also comprises tert-Butyloxycarbonyl protected amine (BocHN);
R₂ is selected from group comprising of OMe, Me, CN, Methyl, Cl, F, Br, NO₂, OPh, OTs, allylic and the like;

In Formula 5,
X is selected from O, N, S
Y is selected from O, N, S
Ar₁ is selected from p-CF₃—C₆H₅, naphthyl
Ar₂ is selected from p-CF₃—C₆H₅, naphthyl
R₂ is selected from the group comprising of OMe, Me, CN, Methyl, Cl, F, Br, NO₂, OPh, OTs, allylic and the like;

In one aspect of the present invention, in Formula 1 the compounds comprising of:

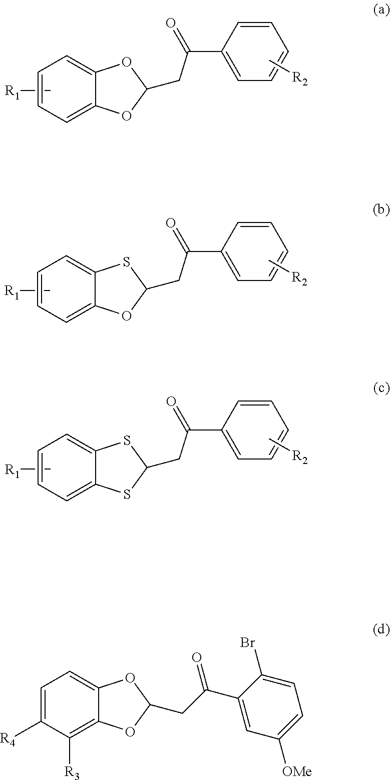

wherein,
R₁ is selected from group comprising of hydro, alkyl, branched chain alkyl, Aryl, tert-butyl, methyl, cyano, o-methyl, nitro and also comprises tert-Butyloxycarbonyl protected amine (BocHN);
R₂ is selected from group comprising of OMe, Me, CN, Methyl, Cl, F, Br, NO₂, OPh, OTs, allylic and the like,
R₃ is selected from the group comprising of Me, H, and
R₄ is selected from the group comprising of Me, H.

In one aspect of the present invention, in formula 3 is the compound comprising formula (e):

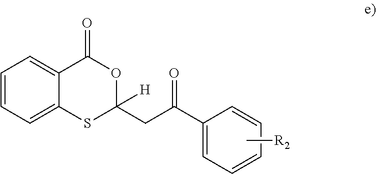

wherein, R₂ is selected from group comprising of OMe, Me, CN, Methyl, Cl, F, Br, NO₂, OPh, OTs, allylic and the like.

In one aspect of the present invention, in formula 5 is the compound represented by (f) comprising:

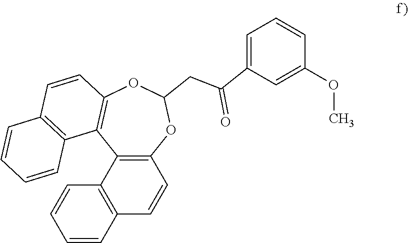 [SMILES: COc1cccc(C(=O)CC2Oc3ccc4ccccc4c3-c3c(ccc4ccccc34)O2)c1]

In one aspect of the present invention, the plurality of monomers is selected from the group of triethylene glycol dimethacrylate (TEGMA), diurethane dimethacrylate (UDMA), bisphenol A-glycidyl methacrylate (Bis-GMA), hexamethylene and the like.

In one aspect of the present invention, the plurality of monomers comprises a first monomer UDMA in the concentration range of 60 wt %-80 wt % and a second monomer TEGMA in the concentration range of 20 wt %-40 wt %.

In one aspect of the present invention, the photo initiator is camphorquinone in the concentration range of 0.1 mol %-2.0 mol %.

In one aspect of the present invention, the co-initiator in the range of 0.1 mol %-2.0 mol %.

In one aspect of the present invention, in the formula 1, the compound is selected from the group comprising of
1. 2-(5-(tert-butyl)benzo[d][1,3]dioxol-2-yl)-1-(3-methoxyphenyl) ethan-1-one (1)
2. 2-(4,6-di-tert-butyl benzo[d][1,3]dioxol-2-yl)-1-(3 methoxyphenyl) ethan-1-one (2)
3. 2-(4-methoxybenzo[d][1,3]dioxol-2-yl)-1-(3-methoxyphenyl) ethan-1-one (3)
4. 2-(2-(3-methoxyphenyl)-2-oxoethyl)benzo[d][1,3]dioxole-5-carbonitrile (4)
5. 1-(3-methoxyphenyl)-2-(5-methylbenzo[d][1,3]dioxol-2-yl) ethan-1-one (5)
6. 1-(3-methoxyphenyl)-2-(4-methylbenzo[d][1,3]dioxol-2-yl) ethan-1-one (6)
7. 1-(3-methoxyphenyl)-2-(5-nitrobenzo[d][1,3]dioxol-2-yl) ethan-1-one (7)
8. 1-(2-bromo-5-methoxyphenyl)-2-(5-methylbenzo[d][1,3]dioxol-2-yl) ethan-1-one (8)
9. 1-(2-bromo-5-methoxyphenyl)-2-(4-methoxybenzo[d][1,3]dioxol-2-yl) ethan-1-one (9)
10. 2-(4-methoxybenzo[d][1,3]dioxol-2-yl)-1-(2-nitrophenyl) ethan-1-one (10)
11. 2-(benzo[d][1,3]dioxol-2-yl)-1-(3-methoxyphenyl) ethan-1-one (11)
12. 2-(benzo[d][1,3]oxathiol-2-yl)-1-(3-methoxyphenyl) ethan-1-one (12)
13. 2-(benzo[d][1,3]dithiol-2-yl)-1-(2-nitrophenyl) ethan-1-one (13)
14. 2-(benzo[d][1,3]dithiol-2-yl)-1-(3-methoxyphenyl) ethan-1-one (14)

In one aspect of the present invention, in the formula 3, the compound is selected from the group comprising of
1. 2-(2-(3-methoxyphenyl)-2-oxoethyl)-4H-benzo[d][1,3]oxathiin-4-one (15)
2. 2-(2-(3,4-dimethoxyphenyl)-2-oxoethyl)-4H-benzo[d][1,3]oxathiin-4-one (16)
3. 2-(2-oxo-2-(p-tolyl)ethyl)-4H-benzo[d][1,3]oxathiin-4-one (17)
4. 4-(2-(4-oxo-4H-benzo[d][1,3]oxathiin-2-yl) acetyl)benzonitrile (18)
5. 2-(2-(4-methoxyphenyl)-2-oxoethyl)-4H-benzo[d][1,3]oxathiin-4-one (19)
6. 2-(2-oxo-2-(3,4,5-trimethoxyphenyl)ethyl)-4H-benzo[d][1,3]oxathiin-4-one (20)
7. 2-(2-oxo-2-phenylethyl)-4H-benzo[d][1,3]oxathiin-4-one (21)
8. 2-(2-(4-chlorophenyl)-2-oxoethyl)-4H-benzo[d][1,3]oxathiin-4-one (22)
9. 2-(2-(4-fluorophenyl)-2-oxoethyl)-4H-benzo[d][1,3]oxathiin-4-one (23)
10. 2-(2-(4-bromophenyl)-2-oxoethyl)-4H-benzo[d][1,3]oxathiin-4-one (24)
11. 2-(2-(4-nitrophenyl)-2-oxoethyl)-4H-benzo[d][1,3]oxathiin-4-one (25)
12. 2-(2-(4-(allyloxy)phenyl)-2-oxoethyl)-4H-benzo[d][1,3]oxathiin-4-one (26)
13. 2-(2-oxo-2-(3-phenoxyphenyl)ethyl)-4H-benzo[d][1,3]oxathiin-4-one (27)
14. 2-(2-(6-bromobenzo[d][1,3]dioxol-5-yl)-2-oxoethyl) 4Hbenzo[d][1,3]oxathiin-4-one (28)
15. 2-(2-(2-bromo-5-methoxyphenyl)-2-oxoethyl)-4H-benzo[d][1,3]oxathiin-4-one (29)
16. 4-(2-(4-oxo-4H-benzo[d][1,3]oxathiin-2-yl) acetyl)phenyl4-methyl benzenesulfonate (30)
17. 2-(2-(2-bromophenyl)-2-oxoethyl)-4H-benzo[d][1,3]oxathiin-4-one (31)

In one aspect of the present invention the compound of Formula 4, is selected from:
1. 2-(2-cyclopropyl-2-oxoethyl)-4H-benzo[d][1,3]oxathiin-4-one (32)
2. 2-(2-oxo-4-phenylbutyl)-4H-benzo[d][1,3]oxathiin-4-one (33)
3. 2-(2-(cyclohex-2-en-1-yl)-2-oxoethyl)-4H-benzo[d][1,3]oxathiin-4-one (34)
4. 2-(2-cyclohexyl-2-oxoethyl)-4H-benzo[d][1,3]oxathiin-4-one (35)

In one aspect of the present invention, in the formula 5, the compound is selected from
1. 2-(dinaphtho[2,1-d: 1′,2′-f][1,3]dioxepin-4-yl)-1-(3 methoxyphenyl) ethan-1-one (36)

In another aspect of the present invention the method of preparing a dental composite, comprising the steps of:
i. reacting a first monomer with a second monomer along with a photo initiator and a co-initiator selected from Formula 1 or Formula 2 or Formula 3 or Formula 4 or Formula 5 in the presence of Blue LED light and at RT or room temperature to yield a dental composite mixture; thereby polymeric reacted composite substance is obtained
ii. subjecting the dental composite mixture in the step (i) to light source for a time period of 25-40 seconds resulting in polymerization of the dental composite for dental restoration.

In another aspect of the present invention, the first monomer UDMA in the concentration range of 60 wt %-80 wt %.

In another aspect of the present invention, the second monomer TEGMA in the concentration range of 20 wt %-40 wt %.

In another aspect of the present invention, the photo initiator is camphorquinone in the concentration range of 0.1 mol %-2.0 mol %.

In another aspect of the present invention, the light source is Blue LED light source which is in the range of 10W-20W.

In another aspect of the present invention, the wavelength of light source is in the range of 450 nm.

In yet another aspect of the present invention, the co-initiator is in the range of 0.1 mol %-2.0 mol %.

In further aspect of the present invention, the generated polymer sustains high glass transition temperature T_{g} 97.7±1° C.

### BRIEF DESCRIPTION OF THE DRA WINGS

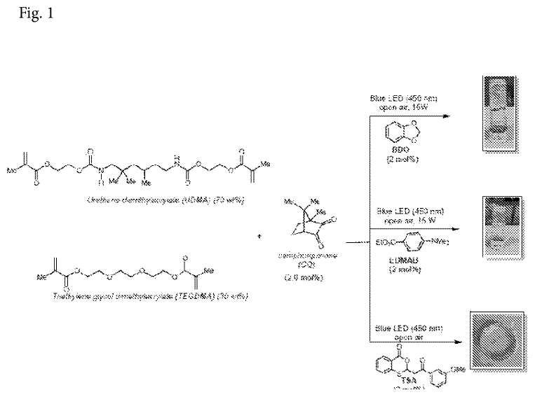

FIG. 1 illustrates the comparative study of different co-initiators BDO, EDMAB and TSA with respect to polymerization.

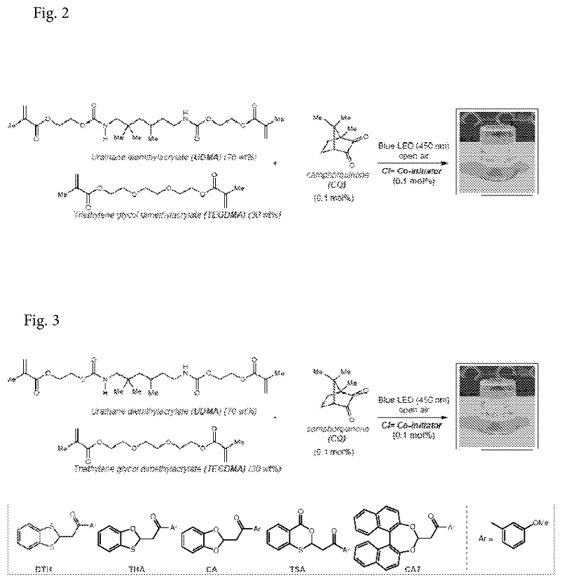

FIG. 2 illustrates the optimization of co-initiator loading.

FIG. 3 illustrates screening of different co-initiator and different light source (power).

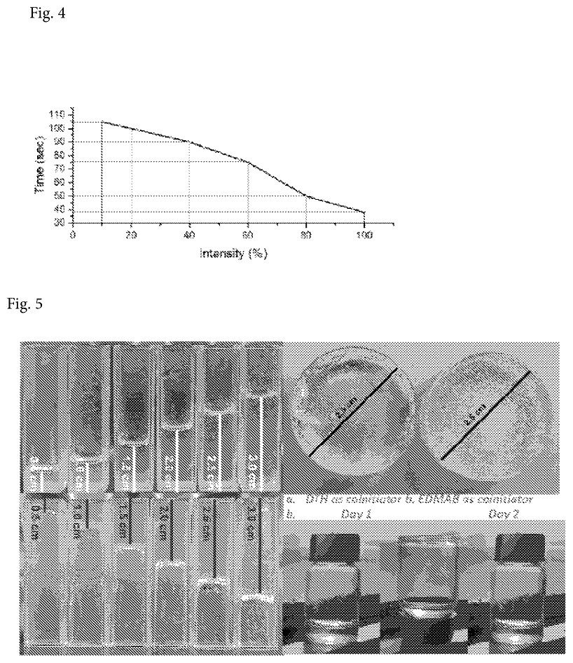

FIG. 4 illustrates effect of intensity of light on polymerization reaction of UDMA and TEGDMA.

FIG. 5 (5A) illustrates in depth polymerization by DTH, (5B) extent of coloration during the polymerization using DTH and EDMAB as co-initiator and (5C) It also illustrates the necessity of blue light for polymerization.

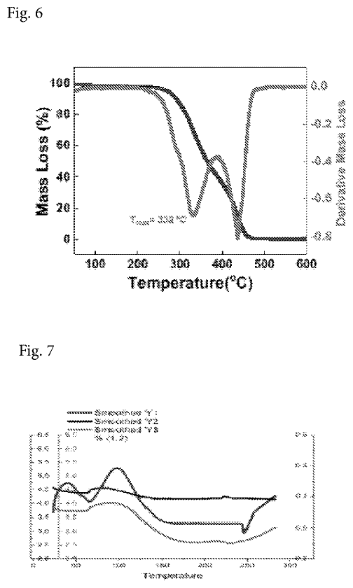

FIG. 6 illustrates the graphical representation of Mass Loss (%) Vs Temperature (° C.).

FIG. 7 illustrates the graphical representation Rheological Data.

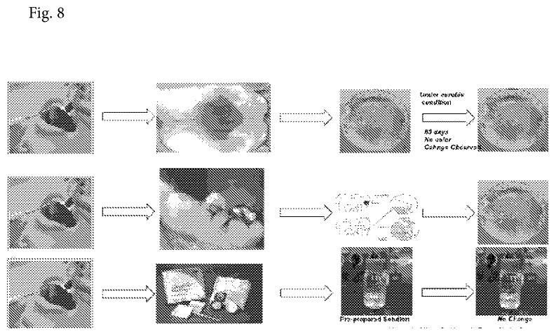

FIG. 8 illustrates the control experiment for color change under aerobic conditions.

### DETAILED DESCRIPTION OF THE INVENTION

The present invention as embodied by “A DENTAL COMPOSITE AND METHOD OF PHOTO-INDUCED POLYMERISATION USING NOVEL CO-INITIATOR” succinctly fulfils the above-mentioned need(s) in the art. The present invention has objective(s) arising as a result of the above-mentioned need(s), said objective(s) being enumerated below. In as much as the objective(s) of the present invention are enumerated, it will be obvious to a person skilled in the art that the enumerated objective(s) are not exhaustive of the present invention in its entirety, and are enclosed solely for the purpose of illustration. Further, the present invention encloses within its scope and purview, any structural alternative(s) and/or any functional equivalent(s) even though such structural alternative(s) and/or any functional equivalent(s) are not mentioned explicitly herein or elsewhere, in the present disclosure. The present invention therefore encompasses also, any improvisation(s)/modification(s) applied to the structural alternative(s)/functional alternative(s) within its scope and purview. The present invention may be embodied in other specific form(s) without departing from the spirit or essential attributes thereof.

Throughout this specification, the use of the word “comprise” and variations such as “comprises” and “comprising” may imply the inclusion of an element or elements not specifically recited.

The present invention in various embodiments discloses a dental composite, comprising photo induced polymerization reaction product. The process of polymerization has three phases: initiation, propagation and termination. Free radicals are necessary to lengthen the chain of polymer and they are formed by photo initiators. The photo induced polymerization reaction product is a result of a reaction between one or more monomers, a photo initiator, and a co-initiator. The reaction takes place at room temperature. The process and the reaction products as embodied by various embodiments of the present invention are discussed in detail below.

Present invention is based on photo induced polymerization reaction which is a process when monomers react together to convert into polymers. The monomers used in dentistry: Bis-GMA, TEGMA, UDMA etc., are liquids and as a result of polymerization they become solids. The process of polymerization has three phases: initiation, propagation and termination. Free radicals are necessary to lengthen the chain of polymer and they are formed by photo initiators. In the present invention extensive research is performed on light cured photo induced polymerization reaction which is disclosed in great detail of the description below. The light cured dental composite of the present invention finds its application as a filling material in dental restoration procedures. The dental composite is a photo induced polymerization reaction product. Monomers used in dentistry are: Bis-GMA, TEGMA, UDMA etc., are liquids and as a result of polymerization they become solids. In exemplary embodiments of the present invention are chosen. The reaction occurs at room temperature.

In one embodiment of the present invention the plurality of monomers is selected from the group comprising of triethylene glycol dimethacrylate (TEGMA), diurethane dimethacrylate (UDMA), bisphenol A-glycidyl methacrylate (BisGMA), hexamethylene and the like, in which the monomers are segregated as first monomer and second monomer.

In exemplary embodiment of the present invention the dental composite comprises a combination of two monomers comprising, a first monomer diurethane dimethacrylate (UDMA), employed in a concentration range of 60 wt %-80 wt % and a second monomer triethylene glycol dimethacrylate (TEGMA) employed in a range of 20 wt %-40 wt %.

The present invention is based on type-II photoinitiators (e.g. camphorquinone (CQ), phenanthrenequinone (PQ), benzophenone (BP) and 1-phenyl-1,2propanodione (PPD)) which initiate the polymerization by abstraction of hydrogen atom from the co-initiator. This invention explored the use of photo initiators ranging from camphorquinone (CQ), phenanthrenequinone (PQ), benzophenone (BP) with co-initiators embodied by compounds of Formula 1, formula 2, Formula 3, Formula 4 and Formula 5 of the present invention. The photo initiators of the present invention are selected such that their absorption band lies between 400-490 nm. The polymerization initiates by photons of visible blue light. The co-initiators of CQ are mostly aromatic tertiary amines. The effective concentration of CQ and co-initiators is obtained to gain a high degree of conversion. The polymerization is initiated by irradiation of CQ by blue light and it goes to the triplet state from singlet state. The radical formation is highly dependent upon the co-initiator type, the concentration and its structure.

In one of the preferred embodiments, the present invention shall disclose a dental composite, comprising photo induced polymerization reaction product of
i. a plurality of monomers,
ii. a photo initiator, and
iii. a co-initiator,
wherein the co-initiator is selected from the compounds of Formula 1, Formula 2, Formula 3, Formula 4 and Formula 5 In Formula 1 and Formula 3,

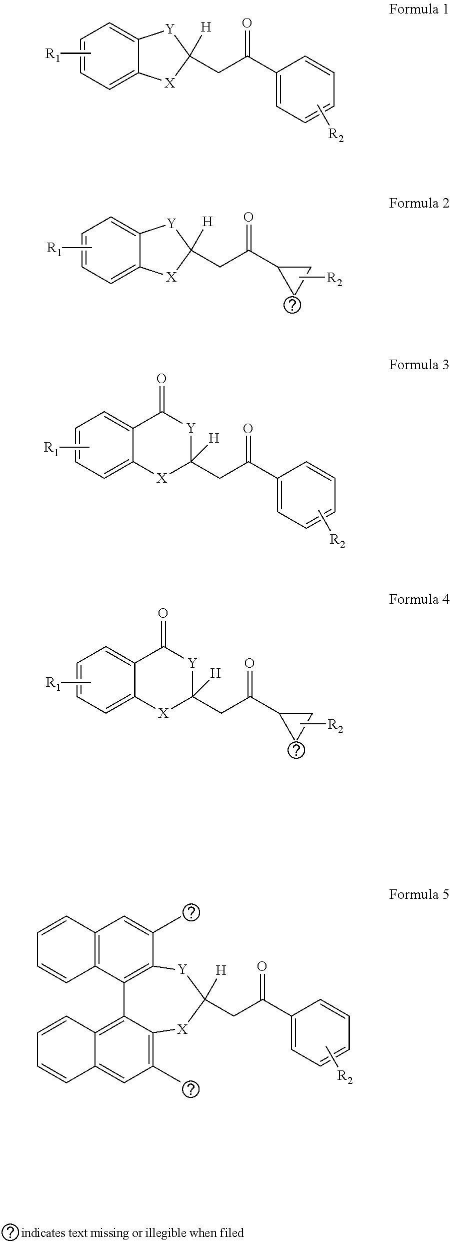

X is selected from O, S, N, Se and Te;
Y is selected from O, S, N, Se and Te;
R₁ is selected from the group comprising of Hydro, alkyl, branched chain alkyl, aryl, t-butyl, methyl, cyano, o-methyl, nitro and also comprises tert-butyloxycarbonyl amine (NHBoc); R₂ is selected from group comprising of OMe, Me, CN, Methyl, Cl, F, Br, NO₂, OPh, OTs, allylic and the like;

In Formula 2 and Formula 4,
X is selected from O, S, N, Se and Te;
Y is selected from O, S, N, Se and Te;
wherein n=0, 1, 2, 3, 4, 5, 6
R₁ is selected from the group comprising of Hydro, alkyl, branched chain alkyl, aryl, tert-butyl, methyl, cyano, o-methyl, nitro and also comprises tert-butyloxycarbonyl protected amine (BocHN);
R₂ is selected from group comprising of OMe, Me, CN, Methyl, Cl, F, Br, NO₂, OPh, OTs, allylic and the like;

In Formula 5,
X is selected from O, N, S
Y is selected from O, N, S
Ar₁ is selected from p-CF₃—C₆H₅, naphthyl
Ar₂ is selected from p-CF₃—C₆H₅, naphthyl
R₂ is selected from the group comprising of OMe, Me, CN, Methyl, Cl, F, Br, NO₂, OPh, OTs, allylic and the like;

Moreover, monomers are selected from triethylene glycol dimethacrylate (TEGMA), diurethane dimethacrylate (UDMA) are commonly used monomeric unit in dental restoration process.

In dental composite of the present invention list of photoinitiators are employed for experimental analysis such as benzil, 1-phenylpropane-1,2-dione, 2-methoxy-1,2-diphenlethan-1-one, thioxanthene-9-one, anthracene-9,10-dione, 2,2-dimethoxy-2-phenylacetophenone. Among these initiators camphorquinone turned out to be the best initiator in the visible range.

The photo initiator selected as is camphorquinone which is employed in a concentration range of 0.1 mol %-2.0 mol %.

The co-initiator is selected from the compounds of Formula (1), Formula (2), Formula (3), Formula (4) and Formula (5), wherein;
X is selected from O, S, N, Se and Te;
Y is selected from O, S, N, Se and Te;
R₁ is selected from group comprising of CH₂—CO—Ar—R₂ in which R is selected from Hydro, alkyl, branched chain alkyl, aryl, t-butyl, methyl, cyano, o-methyl, nitro and also comprises tert-butyloxycarbonyl protected amine (BocHN) and
R₂ is selected from group comprising of ortho, meta, para positioned OMe, Me, CN, Methyl, Cl, F, Br, NO₂, OPh, OTs and the like.

The dental composite of a preferred embodiment of the present invention, as mentioned above the co-initiator is selected from the compounds of Formula (1), includes, the group of

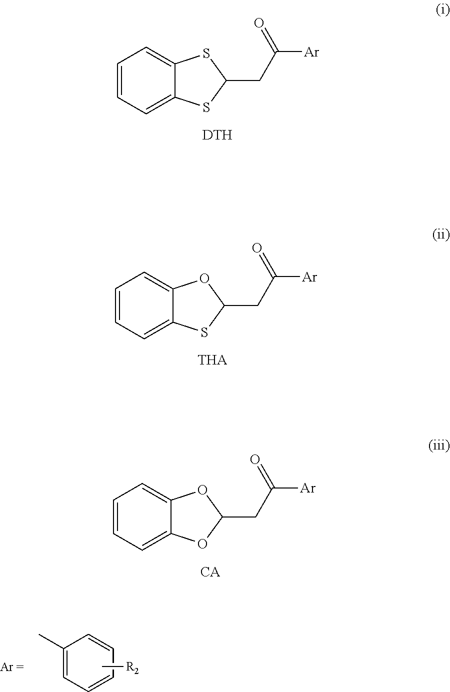

R₂ is selected from the group comprising of ortho, meta, para positioned OMe, Me, CN, Methyl, CI, F, Br, NO₂, OPh, OTs and the like.

The dental composite of the preferred embodiment of the present invention, as mentioned above the co-initiator is selected from 6,6-bicyclic scaffold compound of Formula (3), which is TSA.

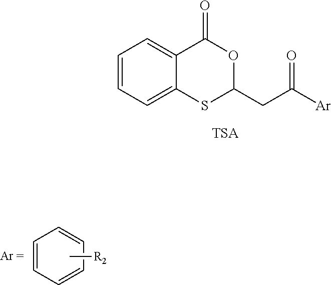

R₂ is selected from the group comprising of ortho, meta, para positioned OMe, Me, CN, Methyl, Cl, F, Br, NO₂, OPh, OTs and the like.

The dental composite of the preferred embodiment of the present invention, in the Formula 5 of a preferred embodiment is:

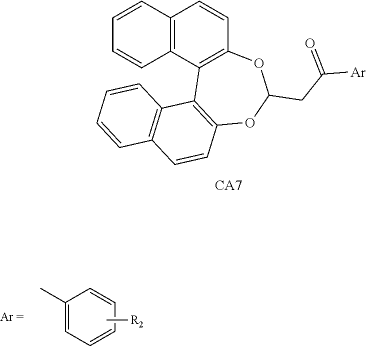

R₂ is selected from the group comprising of ortho, meta, para positioned OMe, Me, CN, Methyl, Cl, F, Br, NO₂, OPh, OTs and the like.

The co-initiator concentration in the present invention is in range of 0.1 mol %-2.0 mol %.

Representative compounds comprising Formula 1, Formula 2, Formula 3, Formula 4 and Formula 5 of preferred embodiment of the present invention are presented below:

### Preparation of Dental Composite

A method of preparing a dental composite, comprising the steps of
a) reacting a first monomer with a second monomeric along with photo initiator and co-initiator of Formula 1 or Formula 2 or Formula 3 or Formula 4 or Formula 5 to yield a dental composite mixture;
b) subjecting the dental composite mixture of step (i) to light source for a time period of 25-40 seconds resulting in polymerization of the dental composite for dental restoration.
c) wherein curing of the prepared dental composite mixture is done blue LED light source which is in range of 10W-20W, the wavelength pass through the light source is in 450 nm, i.e., visible light.

Benzodioxole derivatives (BDO) turned out to be suitable alternative for dental application. Generally, co-initiator loading and polymerization delaying are the major drawbacks for BDO-CQ combination of the prior art. In addition to that certain amount of yellowing was also observed. The present invention provided newly synthesized co-initiators represented by 6,5 and 6,6-bicyclic, 6,7,6-tricyclic scaffolds and to develop a novel dental composite as filling material for dental restoration purposes.

### Example 1

Comparative Study of Different Co-initiator: (FIG. 1): Firstly, to perform control experiment for validating innovative hypothesis EDMAB, BDO and TSA as co-initiator for the polymerization of monomeric composition diurethane dimethacrylate (UDMA, 70 wt %) and triethylene glycol dimethacrylate (TEGMA, 30 wt %) in presence of 450 nm LED (15 W) under the aerobic conditions is taken.

Monomer 1, diurethane dimethacrylate (UDMA, 70 wt %), with monomer 2, triethylene glycol dimethacrylate (TEGMA, 30 wt %) followed by addition of photoinitiator type II, Camphorquinone CQ in the concentration of 2 mol %, further added
(i) 2 mol % BDO and initiation of photo induced polymerization under Blue LED light of 450 nm, resulted in complete polymerization in 4 minutes.
(ii) 2 mol % ethyl 4-(dimethylamino)benzoate (EDMAB) and initiation of photo induced polymerization under Blue LED light of 450 nm, resulted in complete polymerization in 4 minutes.
(iii) Representative compound TSA (2 mol %) and initiation of photo induced polymerization under Blue LED light of 450 nm, resulted in complete polymerization in 4 minutes.

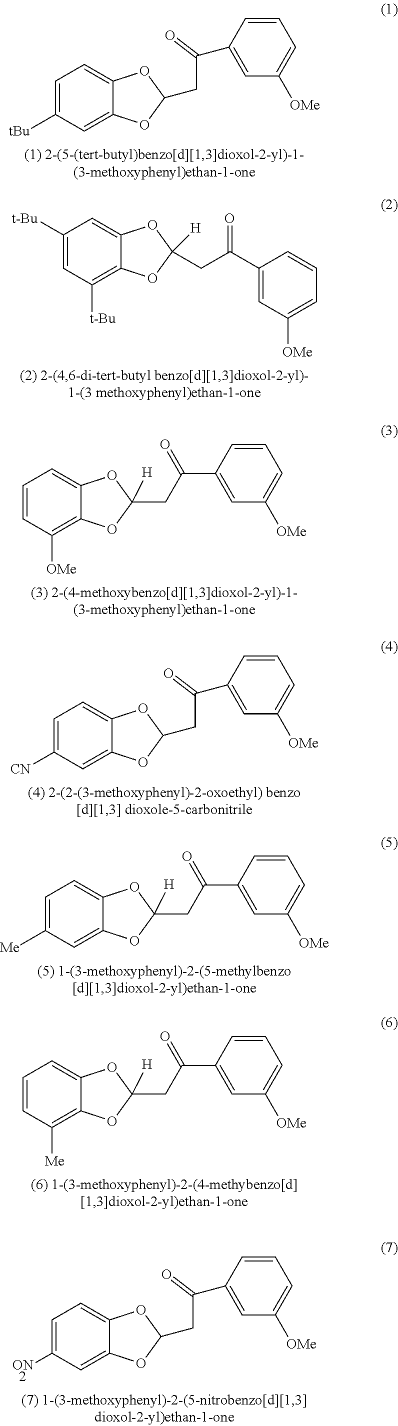

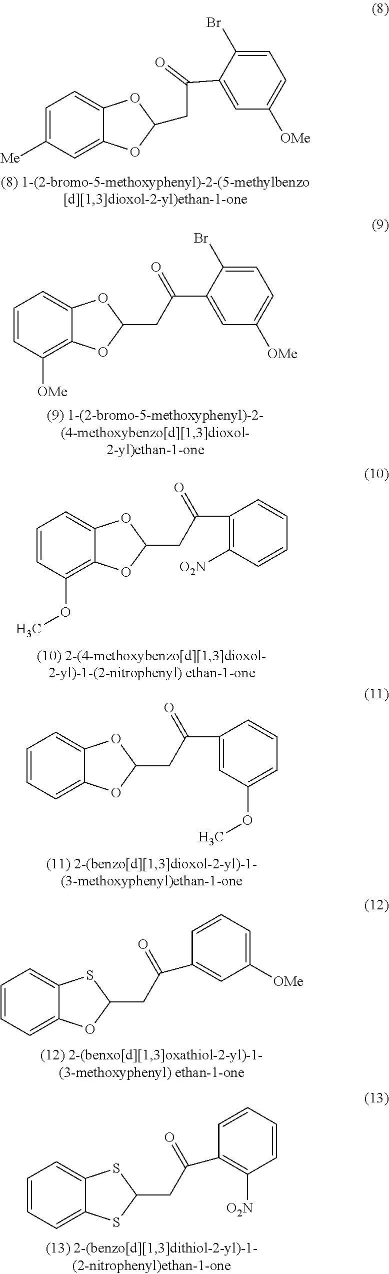 [SMILES: COc1ccc(Br)c(C(=O)CC2Oc3ccc(C)cc3O2)c1 ; COc1ccc(Br)c(C(=O)CC2Oc3cccc(OC)c3O2)c1 ; COc1cccc2c1OC(CC(=O)c1ccccc1[N+](=O)[O-])O2 ; COc1cccc(C(=O)CC2Oc3ccccc3O2)c1 ; COc1cccc(C(=O)CC2Oc3ccccc3S2)c1 ; O=C(CC1Sc2ccccc2S1)c1ccccc1[N+](=O)[O-]]

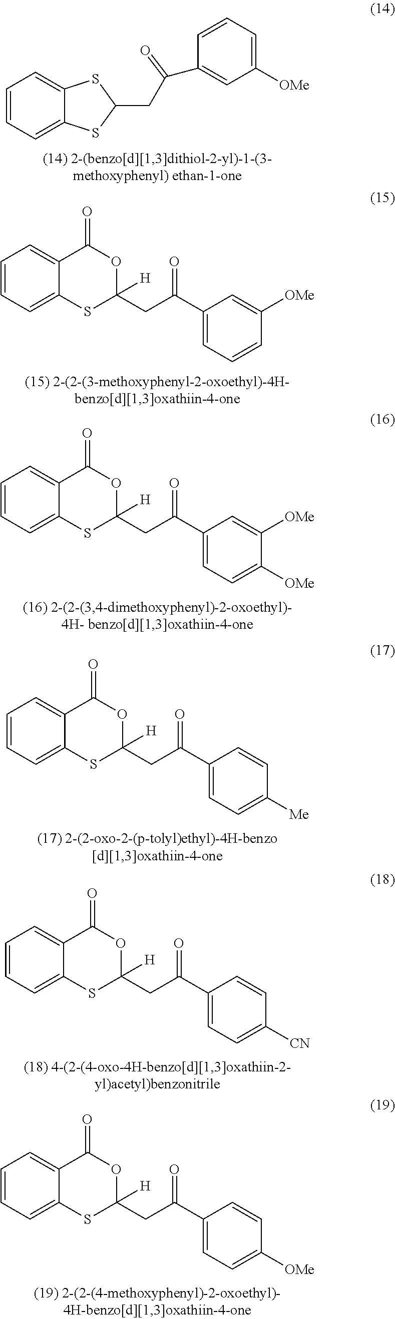 [SMILES: COc1cccc(C(=O)CC2Sc3ccccc3S2)c1 ; COc1cccc(C(=O)CC2OC(=O)c3ccccc3S2)c1 ; COc1ccc(C(=O)CC2OC(=O)c3ccccc3S2)cc1OC ; Cc1ccc(C(=O)CC2OC(=O)c3ccccc3S2)cc1 ; N#Cc1ccc(C(=O)CC2OC(=O)c3ccccc3S2)cc1 ; COc1ccc(C(=O)CC2OC(=O)c3ccccc3S2)cc1]

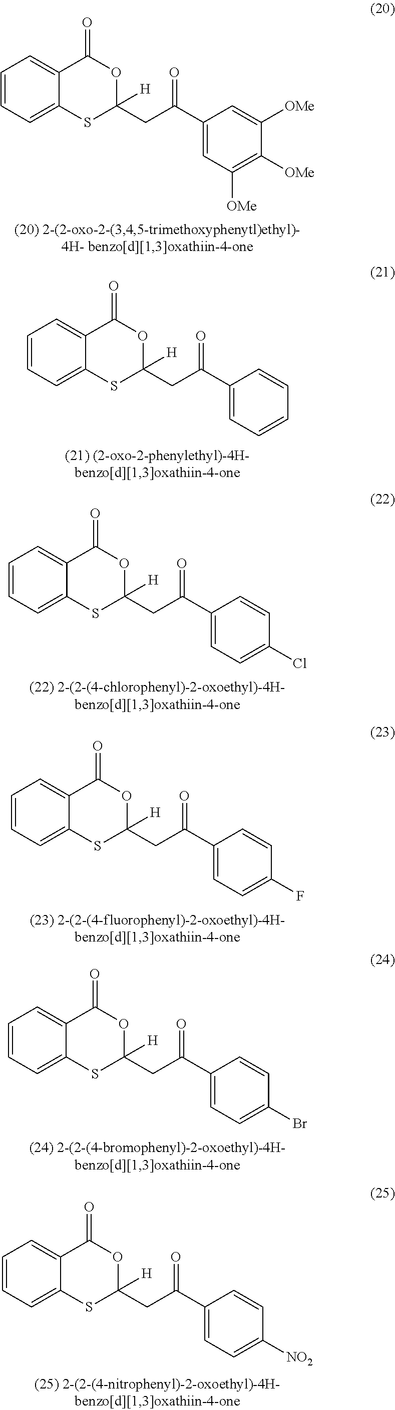 [SMILES: COc1cc(C(=O)CC2OC(=O)c3ccccc3S2)cc(OC)c1OC ; O=C(CC1OC(=O)c2ccccc2S1)c1ccccc1 ; O=C(CC1OC(=O)c2ccccc2S1)c1ccc(Cl)cc1 ; O=C(CC1OC(=O)c2ccccc2S1)c1ccc(F)cc1 ; O=C(CC1OC(=O)c2ccccc2S1)c1ccc(Br)cc1 ; O=C(CC1OC(=O)c2ccccc2S1)c1ccc([N+](=O)[O-])cc1]

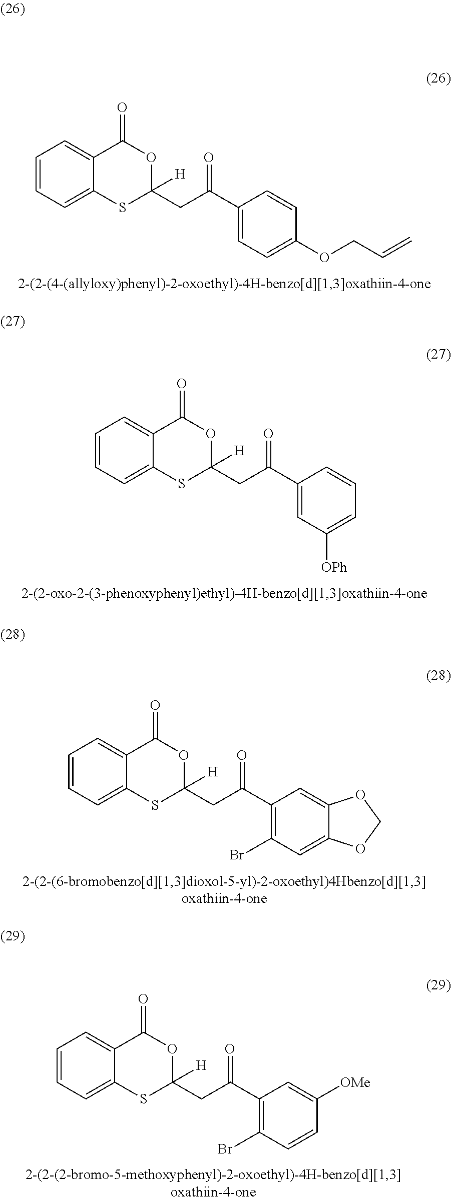 [SMILES: C=CCOc1ccc(C(=O)CC2OC(=O)c3ccccc3S2)cc1 ; O=C(CC1OC(=O)c2ccccc2S1)c1cccc(Oc2ccccc2)c1 ; O=C(CC1OC(=O)c2ccccc2S1)c1cc2c(cc1Br)OCO2 ; COc1ccc(Br)c(C(=O)CC2OC(=O)c3ccccc3S2)c1]

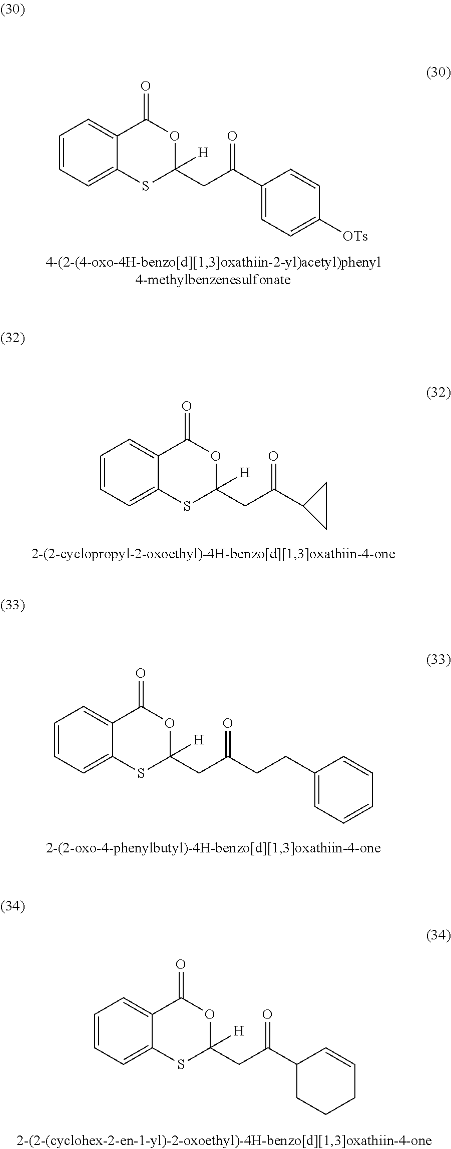 [SMILES: Cc1ccc(S(=O)(=O)Oc2ccc(C(=O)CC3OC(=O)c4ccccc4S3)cc2)cc1 ; O=C1OC(CC(=O)C2CC2)Sc2ccccc21 ; O=C(CCc1ccccc1)CC1OC(=O)c2ccccc2S1 ; O=C1OC(CC(=O)C2C=CCCC2)Sc2ccccc21]

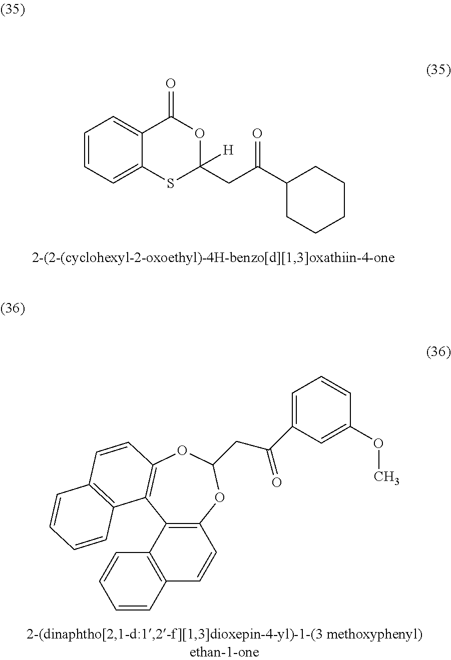 [SMILES: O=C1OC(CC(=O)C2CCCCC2)Sc2ccccc21 ; COc1cccc(C(=O)CC2Oc3ccc4ccccc4c3-c3c(ccc4ccccc34)O2)c1]

As illustrated in FIG. 1, the compound TSA resulted in clear dental composited devoid of any yellowing.

Secondly, the complete polymerization reaction completed within 4 minutes and polymeric materials was completely white, which indicates comparable reactivity and almost no yellowing coloration detected.

### Example 2

Determination of optimum Co-initiator Loading (FIG. 2)

Tested with [UDMA]+ [TEGMA]reacted with a photoinitiator such as CQ and co-initiator [2-(2-(3-methoxyphenyl)-2-oxoethyl)-4H-benzo[d][1,3]oxathiin-4-one (TSA)] of compound 15 of Formula 3 at various concentrations as presented below in Table 2.

TABLE 2
Time for polymerization of the dental composite at different
concentrations of photoinitiator, and co-initiator.

| S. | UDMA | TEGMA | CQ | Co | Light | Time |
|---|---|---|---|---|---|---|
| No. | (wt %) | (wt %) | (mol %) | (mol %) | Source | (min) |
| 1. | 70 | 30 | 2 | 2.0 | 450 nm | 3:10 |
| 2. | 70 | 30 | 2 | 1.5 | 450 nm | 3:09 |
| 3. | 70 | 30 | 2 | 1.0 | 450 nm | 3:10 |
| 4. | 70 | 30 | 2 | 0.5 | 450 nm | 3:10 |
| 5. | 70 | 30 | 2 | 0.4 | 450 nm | 3:08 |
| 6. | 70 | 30 | 2 | 0.3 | 450 nm | 3:08 |
| 7. | 70 | 30 | 2 | 0.2 | 450 nm | 3:08 |
| 8. | 70 | 30 | 2 | 0.1 | 450 nm | 3:08 |
| 9. | 70 | 30 | 1 | 0.1 | 450 nm | 3:08 |
| 10. | 70 | 30 | 0.5 | 0.1 | 450 nm | 3:10 |
| 11. | 70 | 30 | 0.1 | 0.1 | 450 nm | 2:50 |

### Result:

To optimize the polymerization reaction conditions by lowering the loading of both CQ and TSA, and under optimized reaction conditions employing 0.1 mol % of CQ and TSA, complete polymerization was seen in 2 minutes 50 seconds, significantly faster than BDO, which requires 9 minutes 36 seconds under similar conditions. In presence of 0.1 mol % CQ and EDMAB polymerization time required 15 minutes.

### Example 3

Comparative Study of Different co-initiators (FIG. 3)

TABLE 3A
Study of Light intensity at different power wattage
of the blue light using selected co-initiators.

| S. | Light | DTH | THA | TSA | CA | CA7 |
|---|---|---|---|---|---|---|
| No. | Intensity | (ToP) | (ToP) | (ToP) | (ToP) | (ToP) |
| 1. | 15 W | 1:09 min | 1:47 min | 2:50 min | 3:20 min | 3:40 min |
| 2. | 30 W | 1:02 min | 1:36 min | 2:39 min | — | 3:32 min |
| 3. | 45 W | 0:57 min | 1:27 min | 2:09 min | — | 3:13 min |
| 4. | 100 W | 0:52 min | 1:07 min | 1:56 min | — | 2:42 min |

From the above-mentioned experimental analysis having Formula 1, Formula 2 Formula 3, Formula 4 and Formula 5 the results are summarized in table 3. All the catechol derivatives (1 to 9B) are termed as CA, dithiol derivatives (12) are termed as DTH, thioalcohol derivatives (11) are termed as THA, thiosalicyclic acid derivatives are termed as TSA, binol derivatives are termed as CA7. From each of these category following molecules have been selected for evaluation.
i. (2-(benzo[d][1,3]dithiol-2-yl)-1-(3-methoxyphenyl) ethan-1-one) (DTH)
ii. 2-(benzo[d][1,3]oxathiol-2-yl)-1-(3-methoxyphenyl) ethan-1-one (THA)
iii. 2-(2-(3-methoxyphenyl)-2-oxoethyl)-4H-benzo[d][1,3]oxathiin-4-one (TSA)
iv. 2-(benzo[d][1,3]dioxol-2-yl)-1-(3-methoxyphenyl) ethan-1-one (CA)
v. 2-(dinaphtho[2,1-d: 1′,2′-f][1,3]dioxepin-4-yl)-1-(3 methoxyphenyl) ethan-1-one (CA7)

From the Table no.3A it is clearly inferred that, among these co-initiators (2-(benzo[d][1,3]dithiol-2-yl)-1-(3-methoxyphenyl) ethan-1-one/DTH of Formula 1) is proved to be the best (DTH) and it polymerize the composite in 52 seconds in presence of 100 W light.

### Example 4 (FIG. 4)

a. Monomer 1: UDMA (70% by weight)
b. Monomer 2: TEGMA (30% by weight)
c. Photoinitiator: Camphorquinone (0.1 mol %) 5 d. Co-initiator: compound 14 (2-(benzo[d][1,3]dithiol-2-yl)-1-(3-methoxyphenyl) ethan-1-one) as a co-initiator (0.1 mol %).

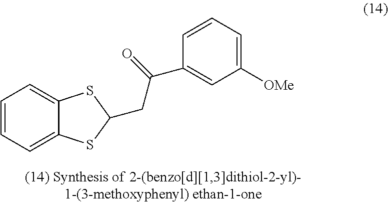 [SMILES: COc1cccc(C(=O)CC2Sc3ccccc3S2)c1]

TABLE 3B
Effect of intensity of light using compound of Formula
14 ((2-(benzo[d][1,3]dithiol-2-yl)-1-(3-methoxyphenyl)
ethan-1-one) as co-initiator.

| S. | UDMA | TEGMA | CQ | Co | Intensity | Time |
|---|---|---|---|---|---|---|
| No. | (wt %) | (wt %) | (mol %) | (mol %) | (%) | (sec) |
| 1. | 70 | 30 | 0.1 | 0.1 | 10 | 105 |
| 2. | 70 | 30 | 0.1 | 0.1 | 20 | 100 |
| 3. | 70 | 30 | 0.1 | 0.1 | 40 | 90 |
| 4. | 70 | 30 | 0.1 | 0.1 | 60 | 75 |
| 5. | 70 | 30 | 0.1 | 0.1 | 80 | 50 |
| 6. | 70 | 30 | 0.1 | 0.1 | 100 | 38 |

Result: From the table 3B it was observed that intensity of the light is playing a crucial role and complete polymerization was observed within short time using 100% of intensity of the light in presence of DTH as co-initiator. With BDO under the same conditions, the polymerization time was 9:36 minutes.

FIG. 4 illustrates the effect of intensity of light in photo-induced polymerization reaction using DTH as co-initiator. According to the observed trend, DTH can polymerize the composite material in 38 seconds using 100% intensity of the light.

### FIG. 5 Illustrates:

5A) the efficiency of polymerization in depth under the reaction's newly developed conditions. Polymerisation of dental material can be performed up to 3 cm, which will be very crucial for bulk filling dental cavity and polymeric material turned out to be white.
5B) Yellowing is a critical issue in dental restoration process using EDMAB promoted PRP reaction, which is further observed in case of EDMAB promoted polymerisation reaction.
5C) Dental composite can be stored under room temperature and polymerisation does not occur in the absence of BLUE light.

FIG. 6 illustrates TGA thermogram of (TSA promoted polymerization material product) at heating rate of 10° C./min in N₂. According to the observed data 5% mass-loss was observed at 271° C. and at 291° C. 10% mass loss was observed. Maximum mass loss was observed at 332° C.

FIG. 7 illustrates that under present reaction conditions generated polymeric material has 97.7±1° C. glass transition temperature turned out to be higher than the EDMAB promoted polymerised product.

FIG. 8 illustrates the effect of light on the polymeric material over time and this figure also illustrate that the role of blue LED light and mixture of UDMA,

TEGMA, CQ (0.1 mol %) and DTH (0.1 mol %) stays liquid under the ambient condition in the absence of blue light.

Advantages of the present invention:
a) Dental composite of the present invention includes a utility availability of blue LED light sources with wave length-flexibility.
b) The present invention also has a utility factor of broad availability of the monomer formulation, solvent less method and requirement of less energy.
c) Dental composite of the present invention is polymerized and employed for bulk filling up to 3 cm.
d) The stability of the generated polymeric material turned out to be colorless and no yellow coloration can be seen over 10 months.

Properties of Co-initiator according to various embodiments of the present invention.

The present invention discloses a dental composite formed with newly developed co-initiator, which was synthesized by sequential conjugate addition reaction that has the following advantages.
a) Operational simplicity,
b) Great atom economy,
c) Ease of purification and bench stability, and
d) Scalability with very high functional group tolerance.
e) A low-cost commercially available starting point
f) Reaction at room temperature
g) Application in Photo Induced Polymerization Reaction

The best co-initiator for photo-induced radical polymerization was a benzodithiol-based co-initiator (molecule 14). It took 38 seconds to polymerize the dental composite composed of 70 wt % UDMA and 30 wt % TEGMA using a 15W blue LED light.

Although, the following benefits are associated with this photo induced radical polymerization process hence, no solvent required, the process can be carried out in the open air. It is extremely hydrophilic, high glass transition temperature. Polymeric materials are colorless and do not fade over time, these properties make the present invention more efficient, resourceful and economic. Thus, the present invention discloses an innovative dental composite.

Although the invention has now been described in terms of certain preferred embodiments and exemplified with respect thereto, one skilled in art can readily appreciate that various modifications, changes, omissions and substitutions may be made without departing from the spirit thereof. It is intended therefore that the present invention be limited solely by the scope of the following claims.

1. A dental composite, comprising photo induced polymerization reaction product of:
i. a plurality of monomers,
ii. a photo initiator, and
iii. a co-initiator,
wherein the co-initiator is selected from the compounds of Formula 1, Formula 2, Formula 3, Formula 4, and Formula 5 wherein in Formula 1 and Formula 3, 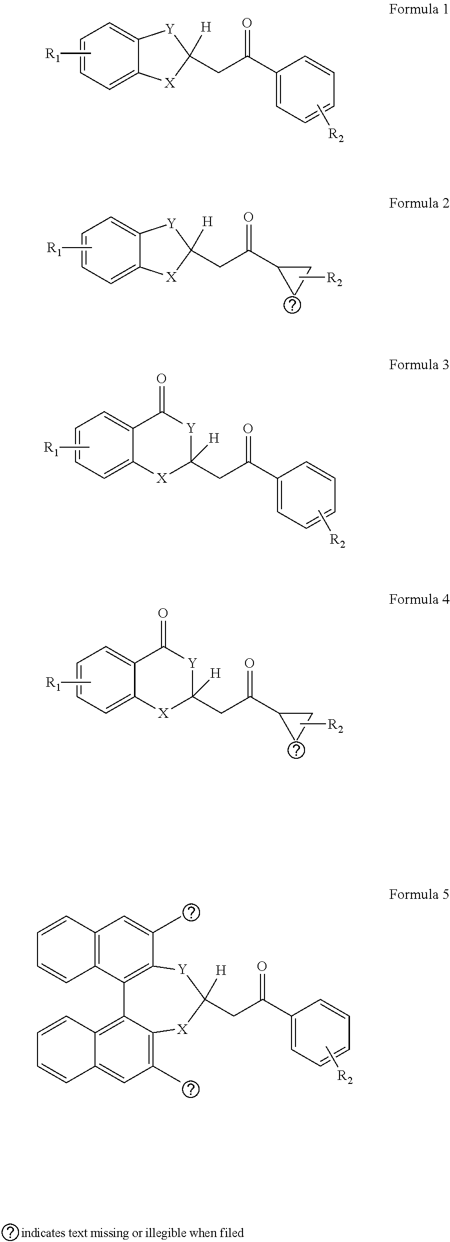
X is selected from O, S, N, Se and
Te; Y is selected from O, S, N, Se and Te;
R₁ is selected from the group comprising of Hydro, alkyl, branched chain alkyl, aryl, t-butyl, methyl, cyano, o-methyl, nitro and also comprises tert-butyloxycarbonyl protected amine (BocHN);
R₂ is selected from group comprising of OMe, Me, CN, Methyl, Cl, F, Br, NO₂, OPh, OTs, allylic and the like;
In Formula 2 and Formula 4,
X is selected from O, S, N, Se and Te,
Y is selected from O, S, N, Se and
Te; wherein n=0, 1, 2, 3, 4, 5, 6;
R₁ is selected from the group comprising of Hydro, alkyl, branched chain alkyl, aryl, tert-butyl, methyl, cyano, o-methyl,
nitro and also comprises tert-butyloxycarbonyl protected amine (BocHN);
R₂ is selected from group comprising of OMe, Me, CN, Methyl, Cl, F, Br, NO₂, OPh, OTs, allylic and the like;
In Formula 5,
X is selected from O, N,
S; Y is selected from O,
N, S;
Ar₁ is selected from p-CF₃—C₆H₅,
naphthyl; Ar₂ is selected from p-CF₃-C₆H₅, naphthyl;
R₂ is selected from the group comprising of OMe, Me, CN, and Methyl, Cl, F, Br, NO₂, OPh, OTs, allylic and the like.

2. The dental composite as claimed in claim 1, wherein the said Formula 1 compound comprising of 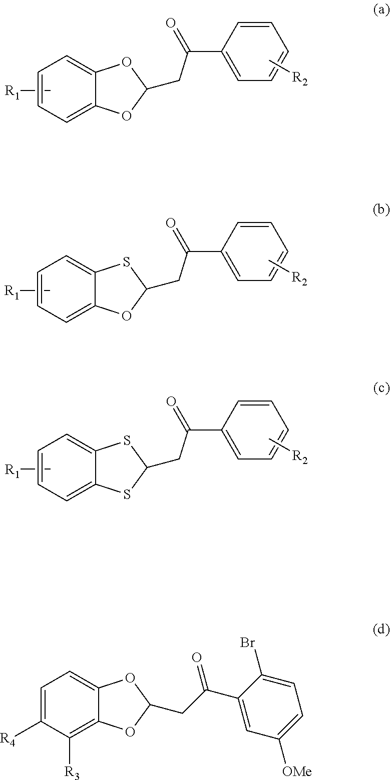
wherein,
R₁ is selected from group comprising of hydro, alkyl, branched chain alkyl, Aryl, tert-butyl, methyl, cyano, o-methyl, nitro and also comprises tert-butyloxycarbonyl protected amine (BocHN);
R₂ is selected from group comprising of OMe, Me, CN, Methyl, Cl, F, Br,
NO₂, OPh, OTs, allylic and the like,
R₃ is selected from the group comprising of Me, H, and R₄ is selected from the group comprising of Me, H.

3. The dental composite as claimed in claim 1, wherein the said formula 3
compound comprising: 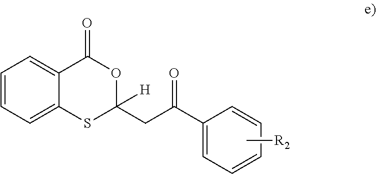
wherein, R₂ is selected from group comprising of OMe, Me, CN, Methyl, Cl, F, Br, NO₂, OPh, OTs, allylic and the like.

4. The dental composite as claimed in claim 1, wherein the said formula 5 is the compound comprising:

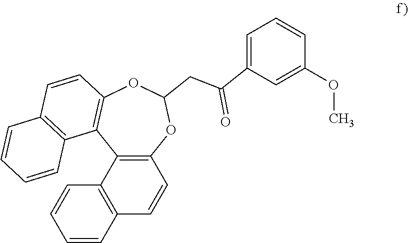 [SMILES: COc1cccc(C(=O)CC2Oc3ccc4ccccc4c3-c3c(ccc4ccccc34)O2)c1]

5. The dental composite as claimed in claim 1, wherein the said plurality of monomers are selected from the group of triethylene glycol dimethacrylate (TEGMA), diurethane dimethacrylate (UDMA), bisphenol A-glycidyl methacrylate (Bis-GMA), hexamethylene and the like.

6. The dental composite as claimed in claim 1,
wherein the
plurality of monomers comprises a first monomer UDMA in the concentration range of 60 wt %-80 wt % and a second monomer TEGMA in the concentration range of 20 wt %-40 wt %.

7. The dental composite as claimed in claim 1, wherein the photo initiator is Camphorquinone in the concentration range of 0.1 mol %-2.0 mol %.

8. The dental composite as claimed in claim 1, wherein the co-initiator in the range of 0.1 mol %-2.0 mol %.

9. The dental composite as claimed in claim 1, wherein in the formula 1 the compound, is selected from the group comprising of
1. 2-(5-(tert-butyl)benzo[d][1,3]dioxol-2-yl)-1-(3-methoxyphenyl) ethan-1-one (1)
2. 2-(4,6-di-tert-butylbenzo[d][1,3]dioxol-2-yl)-1-(methoxyphenyl) ethan-1-one (2)
3. 2-(4-methoxybenzo[d][1,3]dioxol-2-yl)-1-(3-methoxyphenyl) ethan-1-one (3)
4. 2-(2-(3-methoxyphenyl)-2-oxoethyl)benzo[d][1,3]dioxole-5-carbonitrile (4)
5. 1-(3-methoxyphenyl)-2-(5-methylbenzo[d][1,3]dioxol-2-yl) ethan-1-one (5)
6. 1-(3-methoxyphenyl)-2-(4-methylbenzo[d][1,3]dioxol-2-yl) ethan-1-one (6)
7. 1-(3-methoxyphenyl)-2-(5-nitrobenzo[d][1,3]dioxol-2-yl) ethan-1-one (7)
8. 1-(2-bromo-5-methoxyphenyl)-2-(5-methylbenzo[d][1,3]dioxol-2-yl) ethan-1-one (8)
9. 1-(2-bromo-5-methoxyphenyl)-2-(4-methoxybenzo[d][1,3]dioxol-2-yl) ethan-1-one (9)
10. 2-(4-methoxybenzo[d][1,3]dioxol-2-yl)-1-(2-nitrophenyl) ethan-1-one (10)
11. 2-(benzo[d][1,3]dioxol-2-yl)-1-(3-methoxyphenyl) ethan-1-one (11)
12. 2-(benzo[d][1,3]oxathiol-2-yl)-1-(3-methoxyphenyl) ethan-1-one (12)
13. 2-(benzo[d][1,3]dithiol-2-yl)-1-(2-nitrophenyl) ethan-1-one (13)
14. 2-(benzo[d][1,3]dithiol-2-yl)-1-(3-methoxyphenyl) ethan-1-one (14)

10. The dental composite as claimed in claim 1, wherein in the formula 3 the compound, is selected from the group comprising of
1. 2-(2-(3-methoxyphenyl)-2-oxoethyl)-4H-benzo[d][1,3]oxathiin-4-one (15)
2. 2-(2-(3,4-dimethoxyphenyl)-2-oxoethyl)-4H-benzo[d][1,3]oxathiin-4-one (16)
3. 2-(2-oxo-2-(p-tolyl)ethyl)-4H-benzo[d][1,3]oxathiin-4-one (17)
4. 4-(2-(4-oxo-4H-benzo[d][1,3]oxathiin-2-yl) acetyl)benzonitrile (18)
5. 2-(2-(4-methoxyphenyl)-2-oxoethyl)-4H-benzo[d][1,3]oxathiin-4-one (19)
6. 2-(2-oxo-2-(3,4,5-trimethoxyphenyl)ethyl)-4H-benzo[d][1,3]oxathiin-4-one (20)
7. 2-(2-oxo-2-phenylethyl)-4H-benzo[d][1,3]oxathiin-4-one (21)
8. 2-(2-(4-chlorophenyl)-2-oxoethyl)-4H-benzo[d][1,3]oxathiin-4-one (22)
9. 2-(2-(4-fluorophenyl)-2-oxoethyl)-4H-benzo[d][1,3]oxathiin-4-one (23)
10. 2-(2-(4-bromophenyl)-2-oxoethyl)-4H-benzo[d][1,3]oxathiin-4-one (24)
11. 2-(2-(4-nitrophenyl)-2-oxoethyl)-4H-benzo[d][1,3]oxathiin-4-one (25)
12. 2-(2-(4-(allyloxy)phenyl)-2-oxoethyl)-4H-benzo[d][1,3]oxathiin-4-one (26)
13. 2-(2-oxo-2-(3-phenoxyphenyl)ethyl)-4H-benzo[d][1,3]oxathiin-4-one (27)
14. 2-(2-(6-bromobenzo[d][1,3]dioxol-5-yl)-2-oxoethyl) 4Hbenzo[d][1,3]oxathiin-4-one (28)
15. 2-(2-(2-bromo-5-methoxyphenyl)-2-oxoethyl)-7 DOCKET NO. LSDAVP-0010 4H-benzo[d][1,3]oxathiin-4-one (29)
16. 4-(2-(4-oxo-4H-benzo[d][1,3]oxathiin-2-yl) acetyl)phenyl4-methyl benzenesulfonate (30)
17. 2-(2-(2-bromophenyl)-2-oxoethyl)-4H-benzo[d][1,3]oxathiin-4-one (31)

11. The dental composite as claimed in claim 1, wherein in the formula 4, the compound is selected from
1. 2-(2-cyclopropyl-2-oxoethyl)-4H-benzo[d][1,3]oxathiin-4-one (32)
2. 2-(2-oxo-4-phenylbutyl)-4H-benzo[d][1,3]oxathiin-4-one (33)
3. 2-(2-(cyclohex-2-en-1-yl)-2-oxoethyl)-4H-benzo[d][1,3]oxathiin-4-one (34)
4. 2-(2-cyclohexyl-2-oxoethyl)-4H-benzo[d][1,3]oxathiin-4-one (35)

12. The dental composite as claimed in claim 1, wherein in the formula 5, the
compound is selected from
1.2-(dinaphtho[2,1-d: 1′,2′-f][1,3]dioxepin-4-yl)-(3methoxyphenyl) ethan-1-one (36)

13. A method of preparing a dental composite, comprising the steps of:
i. reacting a first monomer with a second monomeric along with a photo initiator and a co-initiator selected from Formula 1, Formula 2, Formula 3, Formula 4 or Formula 5 in the presence of a Blue LED light and at room temperature to yield a dental composite mixture; thereby polymeric reacted composite substance is obtained.
ii. subjecting the dental composite mixture in the step (i) to light source for a time period of 25-40 seconds resulting in polymerization of the dental composite for dental restoration.

14. The dental composite as claimed in claim 13, wherein the first monomer is
UDMA in the concentration range of 60 wt %-80 wt %.

15. The dental composite as claimed in claim 13, wherein the second monomer is TEGMA in the concentration range of 20 wt %-40 wt %.

16. The dental composite as claimed in claim 13, wherein the photo initiator is camphorquinone in the concentration range of 0.1 mol %-2.0 mol %.

17. The dental composite as claimed in claim 13, wherein the light source is Blue LED light source which is in the range of 10W-20W.

18. The dental composite as claimed in claim 13, wherein the wavelength of light source is 450 nm.

19. The dental composite as claimed in claim 13, wherein the co-initiator is in the range of 0.1 mol %-2.0 mol %.

20. The dental composite as claimed in claim 13, wherein the generated polymer sustains high glass transition temperature T_{g} 97.7±1° C.
